(19) United States
(12) Patent Application Publication
(10) Pub. No.: US 2006/0018840 A1
(43) Pub. Date: Jan. 26, 2006

(54) Aerosolizable formulation comprising nicotine

(75) Inventors: David Lechuga-Ballesteros, San Jose, CA (US); Mei-Chang Kuo, Palo Alto, CA (US); Yuan Song, Belmont, CA (US); Blaine Bueche, Foster City, CA (US)

Correspondence Address: NEKTAR THERAPEUTICS, 150 INDUSTRIAL ROAD, SAN CARLOS, CA, 94070, US

(73) Assignee: Nektar Therapeutics, San Carlos, CA (US)

(21) Appl. No.: 11/169,153

(22) Filed: Jun. 28, 2005

Related U.S. Application Data
(60) Provisional application No. 60/583,878, filed on Jun. 28, 2004.

(51) Int. Cl. A61L 9/04; A01N 43/40; A61K 31/4439

(52) U.S. Cl. 424/45; 514/343

(57) ABSTRACT

An aerosolizable formulation comprises free-base nicotine, an organic acid, and a hydrofluoroalkane propellant. The organic acid is present in a mole ratio with said nicotine in a range of about 0.25:1 (organic acid:nicotine) to about 4:1 (organic acid:nicotine). The organic acid and said free-base nicotine form a nicotine salt. An equivalent mixture of free-base nicotine and organic acid in water has a pH between about pH 3.0 and about pH 9.0. The aerosolizable formulation is aerosolizable, for example, by a metered dose inhaler for administration to a user.

This application claims the benefit of U.S. Provisional Application Ser. No. 60/583,878 filed on Jun. 28, 2004, which is incorporated herein by reference in its entirety.

### FIELD OF THE INVENTION

The present invention relates to the delivery of alkaloids, such as nicotine, to the lungs of an individual.

### BACKGROUND

Metered Dose Inhalers (MDIs) comprise a pressure resistant container typically filled with a product, such as an active agent, dissolved or suspended in a liquefied propellant. The pressure resistant container is fitted with a metering valve and an actuator. Actuation of the metering valve aerosolizes and releases a measured dose of the product typically introduced into a subject via inhalation of the aerosol. When actuated, the liquefied propellant aerosolizes the dissolved or micronized drug particles so that they may be delivered to the lungs of an individual during the individual's inhalation.

The administration of aerosol formulations of medicaments using pressurized, MDIs is used widely in therapeutic applications, for example, for treatment of obstructive airway diseases and asthma. Inhalation therapy typically provides more rapid onset of action than oral administration of the same medicament, while minimizing systemic side effects. Aerosol formulations can be administered by inhalation through the mouth or topically by application to the nasal mucosa.

The administration of aerosol formulations via MDIs is dependent upon the propulsive force of the propellant system used in its manufacture. Traditionally, the propellant comprised a mixture of chlorofluorocarbons (CFCs) to provide the desired solubility, vapor pressure, and stability of the formulation. In the past, preferred propellants for use in MDIs were chlorofluorocarbons (commonly called Freons or CFCs) including, but not limited to, CCl₃ F (Freon 11 or CFC-11), CCl₂ F₂ (Freon 12 or CFC-12), and CClF₂—CClF₂ (Freon 114 or CFC-114). Often the propellant used in an MDI was a blend of CFCs. However, the use of chlorofluorocarbons is being phased out because they are considered to be hazardous to the environment. Alternative propellants are increasingly being used in MDIs, for example, environmentally safe hydrofluoroalkane (HFA) propellants or other non-chlorinated propellants.

Various propellants have been suggested for use in MDIs. For example, U.S. Pat. No. 5,182,097 discloses propellant compositions including 1,1,1,2-tetrafluoroethane. European Patent No. EP0372777B1 describes a self-propelling aerosol formulation, which may be free from CFC's, that comprises a medicament, 1,1,1,2-tetrafluoroethane, a surface active agent and at least one compound having a higher polarity than 1,1,1,2-tetrafluoroethane. U.S. Pat. No. 6,419,899 describes a suspension aerosol pharmaceutical formulation for administration of micronized or powdered drug to the respiratory tract of a warm-blooded animal via inhalation comprising 1,1,1,2,3,3,3-heptafluoropropane (HFC-227) and one or more additional propellant gases selected from the group consisting of trichlorofluoromethane, dichlorodifluoromethane, 1,2-dichloro-1,1,2,2-tetrafluoroethane, propane, butane, pentane and dimethylether. U.S. Pat. No. 5,676,930 describes stabilized medicinal aerosol solution formulations comprising medicaments that degrade or decompose by interaction with solvents or water, an HFC propellant, a co-solvent and an acid. The acids (either an inorganic acid or an organic acid) are present in amounts sufficient to reduce the degradation of the medicaments to acceptable levels. U.S. Pat. No. 5,190,029 describes aerosol formulations for use in metered dose inhalers are disclosed which include 1,1,1,2-tetrafluoroethane alone and in combination with other compounds as well as various hydrocarbon blends. U.S. Pat. No. 4,352,789 describes an aerosol composition for dispensing dry particles uniformly in a very fine particle size, comprising solid particles coated with a dry coating of a perfluorinated surfactant, suspended in a propellant (the propellant utilized may be of the perfluorinated environmentally preferred type). U.S. Pat. No. 5,492,688 describes metered dose inhaler formulations that utilize 1,1,1,2-tetrafluoroethane (HFC 134a) as the sole propellant and which include a polar surfactant. U.S. Pat. No. 5,508,023 describes the identification of 1,1,1,2,3,3,3-heptafluoropropane (HFC-227) as a highly polar propellant. The patent describes that surfactants which have an elevated value (9.6 or greater) for their hydrophilic-lipophilic balance (HLB) can be used as suspending, wetting, and lubricating agents or co-solvents in metered dose inhaler formulations pressurized with HFC-227 or propellant blends that contain HFC-227. U.S. Pat. No. 5,182,097 describes aerosol formulations for use in metered dose inhalers that include 1,1,1,2-tetrafluoroethane alone and in combination with other compounds as well as various hydrocarbon blends. All of these references are incorporated herein by reference in their entireties.

Nicotine is the most widely distributed of the plant alkaloids and occurs in two separate phyla of the plant kingdom (Pteridophytes and Spermatophytes). For practical purposes, nicotine is obtained from the tabacum and rustica species of the Nicotina genus. Nicotine can be isolated as an extremely volatile base that has a sharp burning taste. Nicotine can be introduced into the body in many ways with the most popular method being smoking cigarettes. As a cigarette is smoked, the partial oxidation of the tobacco results in the vaporization of some of the nicotine content of the tobacco. Upon inhalation of cigarette smoke, the nicotine vapor, as well as nicotine adsorbed on partial oxidation products of the cigarette, is quickly absorbed through the lungs. After inhalation, nicotine is transported from the lungs to the brain typically in less than 20 seconds.

In recent years there has been a growing recognition of the harmful effects of tobacco smoking. There have been numerous campaigns and programs by governmental agencies and various health groups to disseminate information about the adverse health effects resulting from tobacco smoking. As a result, there have been many programs directed to attempts in reducing smoking incidence. Success in achieving reduction in the incidence of smoking has been relatively poor using presently known techniques and compositions (e.g., behavioral approaches and pharmacological approaches). A high percentage of tobacco smokers who initially quit smoking after using some behavioral or pharmacological approach generally relapse and return to the habit of smoking at their former rate, typically within about a one year's period of time. Even in view of the adverse effects of smoking, nicotine has been used as a successful therapeutic compound, for example, suppressing appetite or preventing weight gain, treatment of neurological disorders, and used as an anti-inflammatory.

Nicotine therapies that do not rely on tobacco are increasingly relied upon to assist in the reduction of the incidence of smoking. A number of such approaches have been described using, for example, nicotine-containing gum, and lozenges/tablets, dental floss, lollypops, transdermal administration, nasal solutions and a variety of inhalation-type devices. PCT International Publication No. WO0105459A1, which is incorporated herein by reference in its entirety, describes a general method to gradually reduce amounts of nicotine delivered to a patient over time, thereby allowing the patient to be gradually weaned off of dependence on nicotine and quit smoking. The system is comprised of a means for aerosolizing a formulation and containers of formulation. The patient uses containers with the highest concentration initially and gradually moves towards using containers with lower and lower concentrations of nicotine until the patient's dependence on nicotine is eliminated. Andrus, P. G., et al., (Can Respir J 6(6):509-512, 1999), which is incorporated herein by reference in its entirety, describe a nicotine microaerosol inhaler. The reference describes the measurement of the droplet size distribution of a nicotine pressurized metered dose inhaler using nicotine in ethanol solution formulation with hydrofluoroalkane as propellant. This reference, however, describes the use of free-base nicotine resulting in a formulation having a highly basic pH.

The prior attempts to provide nicotine delivery methods that avoid the negative effects of tobacco have had limited effectiveness, either in terms of effectiveness or user satisfaction. Thus, there is a need for a method of delivering nicotine in a manner that is safe and effective. It is further desirable to deliver the nicotine in a manner that is satisfactory to the user. It is further desirable to be able to deliver the nicotine in a manner that simulates the nicotine delivery of a cigarette.

### SUMMARY

The present invention satisfies these needs. In one aspect of the invention a pharmaceutical formulation comprises an alkaloid, such as nicotine, in aerosolizable form for administration to a user's respiratory tract.

In another aspect of the invention, an aerosolizable formulation comprises free-base nicotine; an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0; and a hydrofluoroalkane propellant.

In another aspect of the invention, an aerosolization apparatus comprises a canister comprising an aerosolizable formulation comprising nicotine, said formulation being under pressure; a metering valve, and an actuator. In one version, the aerosolizable formulation comprises free-base nicotine; an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0; and a hydrofluoroalkane propellant.

In another aspect of the invention, a method of manufacturing an aerosolization apparatus for administering aerosolized nicotine to a user comprises combining to form an aerosolizable formulation: (i) free-base nicotine, (ii) an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0, and (iii) a hydrofluoroalkane propellant; filling a canister under pressure with an appropriate amount of said aerosolizable formulation; and sealing said canister.

In another aspect of the invention a method of treating nicotine addiction in a subject comprises aerosolizing an aerosolizable formulation comprising nicotine; and administering the aerosolized formulation to the respiratory tract of the subject during the subject's inhalation. In one version, the aerosolizable formulation comprises free-base nicotine; an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0; and a hydrofluoroalkane propellant.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIGS. 1A and 1B show the percent powder mass (vertical axis) deposited on the various stages of an Impactor device using MDIs filled with, respectively, a nicotine free base formulation and a nicotine lactate formulation;

FIG. 2A is a curve showing the relationship between the mole ratio of Acid/Nicotine and pH;

FIG. 2B is a curve showing the relationship between pH and the percent distribution of nicotine for the free base and ionized forms of nicotine;

FIGS. 3A-3C present data for various nicotine lactate formulations plotted for FPD %<4.7 μm, MMAD, and percent throat deposition, respectively, versus percent of ethanol; and

FIG. 4 presents pharmacokinetic data based on a predictive model showing profiles of various forms of nicotine delivery.

### DESCRIPTION

The present invention relates to aerosol drug delivery. Although the process is illustrated in the context of the delivery of alkaloids, such as nicotine, to the lungs of an individual, the present invention can be used in other processes and should not be limited to the examples provided herein.

As used herein, the term “nicotine” refers to the chemical substance commonly referred to as nicotine and having the chemical name S-3-(1-methyl pyrrohdinyl)pyridine in its naturally occurring free-base form, in a salt form, or in any other form. The alkaloid, nicotine, may be isolated and purified from natural sources or synthetically produced. Free-base nicotine is a strong reducing agent, that is, it oxidizes rapidly when exposed to air and reacts chemically with water breaking it down into oxygen and hydrogen. Nicotine (C₁₀H₁₄N₂) is a colorless to pale yellow, strongly alkaline, oily, volatile, hygroscopic liquid having a molecular weight of 162.23. A physiologically active form of nicotine is the S—(−)-isomer. Certain compounds of the present invention may exist in particular geometric or stereoisomeric forms. The present invention contemplates all such compounds, including cis and trans isomers, R and S enantiomers, diastereomers, the racemic mixtures thereof, and other mixtures thereof. Free-base nicotine may be combined with an organic acid (e.g., propionic acid and/or lactic acid) to form a nicotine salt.

As used herein, the term “nicotine” also includes the expression “nicotine and derivatives thereof” which includes any pharmacologically acceptable derivative, metabolite or analog of nicotine that exhibits pharmacotherapeutic properties similar to nicotine. In tobacco, nicotine is typically found with small quantities of nicotimine (C₉H₁₄N₂), nicoteine (C₁₀H₁₂N₂), and nicotelline (C₁₀H₈N₂). Additional asymmetric carbon atoms may be present as a substituent of nicotine, for example, an alkyl group. Further derivatives and metabolites are known in the art, and include, but are not limited to, cotinine (a major metabolite of nicotine), norcotinine, nornicotine, nicotine N-oxide, cotinine N-oxide, 3-hydroxycotinine, and 5-hydroxycofinine. A number of other derivatives of nicotine have been described in, for example, U.S. Pat. Nos. 4,321,387, 4,442,292, 4,965,074, 4,966,916, 5,069,094, 5,138,062, 5,214,060, 5,223,497, 5,227,391, 5,232,933, 5,242,934, 5,276,043, 5,278,045, 5,278,176, 5,721,257, and 5,776,957, all of which are incorporated herein by reference in their entireties. The present invention contemplates use of any such nicotine derivatives and metabolites, as well as combinations thereof, as components of the formulations described herein.

“Alkaloids” as used herein refer to the large family of bitter, alkaline, nitrogenous compounds that typically have pronounced effects on the nervous systems of animals. They may contain phenolic rings or terpenes (steroids). Alkaloids include one of the largest groups of chemicals produced by plants. Alkaloids often contain one or more phenolic or indole rings, usually with a nitrogen atom in the ring. The position of the nitrogen atom in the carbon ring varies with different alkaloids and with different plant families. In some alkaloids, the nitrogen atom is not within a carbon ring. Different families of alkaloids include, but are not limited to, (i) alkaloids with heterocyclic nitrogen atoms (i.e., nitrogen atoms are located within carbon rings), for example, pyridine-piperidine alkaloids (single carbon ring containing one nitrogen atom), tropane alkaloids (contain a methylated nitrogen atom, e.g., scopolamine), isoquinoline alkaloids (double carbon ring containing one nitrogen atom, including narcotic alkaloids commonly found in certain members of the poppy family Papaveraceae, which include morphine, codeine and thebaine), quinolizidine alkaloids (double carbon ring containing one nitrogen atom), indolizidine alkaloids (double ring compounds containing an indole ring), quinoline alkaloids (double carbon ring containing one nitrogen atom), indole alkaloids (double ring compounds containing an indole ring), steroidal alkaloids (double carbon ring containing one nitrogen atom, plus a steroid backbone composed of four carbon rings), purine alkaloids (double carbon ring containing four nitrogen atoms), and muscarine alkaloids (single carbon ring containing oxygen and one nitrogen), (ii) alkaloids without heterocyclic nitrogen atoms (i.e., nitrogen atoms not within a carbon ring, but located in a carbon side chain, e.g., capsaicin), for example, ephedrine alkaloids (amine alkaloids, one or more carbon rings with a nitrogen atom on a carbon side chain), and capsaicin alkaloids.

The term “upper airways” as used herein is used to represent an area of the respiratory system that includes the oropharyngeal region and the trachea.

The term “peripheral region” as used herein represent the region of the respiratory system where gas exchange occurs between the lungs and the circulatory system, that is, the area where oxygen enters the blood and carbon dioxide leaves the blood. Appropriately sized and/or shaped active agents delivered to this area may pass into the blood stream to have a systemic effect. The terms “alveolar ducts” and “alveoli” as used interchangeably herein refer to components located in the peripheral region of the lungs where gas exchange occurs between air in the lungs and the circulatory system.

The terms “central airways” and “bronchial airways” as used herein interchangeably refer to the region of the respiratory system between the upper airways and the peripheral region.

This region includes the bronchial region of the lungs. This area may also be referred to as the “conductive airways” that clean particles from the lung via mucosal clearance. When air is inhaled, it passes through the upper airways into the central airways.

The terms “treatment,” “treating,” and the like as used herein are used interchangeably typically to mean obtaining a desired pharmacological and/or physiological effect. In one version, the treatment methods of the present invention provide the administration of nicotine. For example, a treatment method of the present invention provides a less hazardous mode of inhalation of nicotine than when nicotine is inhaled along with other combustion products from tobacco when the mode of inhalation is cigarette smoking. The desired effect of the treatment may be either the eventual elimination of a user's dependence on nicotine and/or may be merely the delivery of nicotine in a manner that is safer than delivery during inhalation of a tobacco product. Alternatively or additionally, the nicotine may be delivered for the purpose treatment as a therapeutic compound, for example, for suppressing appetite, treatment of neurological disorders, and/or use as an anti-inflammatory.

“Metered Dose Inhaler” (MDI) as used herein refers to an inhalation delivery system typically comprising, for example, a canister containing an active agent dissolved or suspended in a propellant optionally with one or more excipients, a metered dose valve and actuator, and a mouthpiece. As used herein, “mouthpiece” also encompasses a nose piece or any other orifice through which the aerosol may exit the device. The canister is usually filled with a solution or suspension of a compound of interest, such as nicotine, and a propellant, such as one or more hydrofluoroalkanes. The canister, which is often metal, keeps the medication under pressure. When the actuator is depressed a metered dose of the compound is aerosolized for inhalation. Particles comprising the compound of interest are propelled toward the mouthpiece where they may be inhaled by a user. Sometimes, the mouthpiece is in communication with a capture chamber which captures the aerosol for subsequent inhalation by a user in a manner that eliminates the need for the user to coordinate his or her inhalation with the actuation of the device. MDI's are sometimes referred to as pressurized Metered Dose Inhalers, pMDI. When hydrofluoroalkanes are used as propellant, typically the canister is pressurized to prevent vaporization.

“Hydrofluoroalkanes” as used herein generally refer to a halocarbon in which some hydrogen atoms have been replaced by fluorine. Hydrofluoroalkanes (HFAs) are also known also as hydrofluorocarbons (HFCs). HFAs generally contain no chlorine and are considered less destructive to ozone. Exemplary HFAs for medicinal aerosol formulations comprising HFA propellant systems include, but are not limited to, 1,1,1,2-tetrafluoroethane (HFA 134a) and 1,1,1,2,3,3,3-heptafluoropropane (HFA 227), and combinations thereof.

“Organic acid” as used herein refers to an acid made up of molecules containing organic radicals, for example, lactic acid and propionic acid, which contain the ionizable —COOH group. Exemplary organic acids include, but are not limited to, propionic acid, lactic acid, oleic acid, and polyethyleneglycol-propionic acid (PEG-propionate).

“Co-solvent” as used herein refers to a substance, usually a liquid, in which other substances are dissolved. Co-solvents are typically less volatile than propellants and may be used to help dissolve a compound in a propellant, lower the vapor pressure of the propellant system, and/or promote miscibility between propellants and immiscible solvents. Addition of some co-solvents may tend to increase droplet size and wetness. Exemplary co-solvents include, but are not limited to alcohols, e.g. ethanol and isopropylalcohol, and propylene glycol. In the present invention, co-solvents are typically acceptable for pharmaceutical delivery in humans.

The present invention provides a pharmaceutical formulation containing an alkaloid, such as nicotine, in a form suitable for pulmonary administration to a user, such as a human. In one version, the formulation comprises free-base nicotine or a derivative thereof, and an organic acid. The formulation may be formulated to be used in a metered dose inhaler and may comprise a propellant, such as a hydrofluoroalkane propellant, for aerosolizing the formulation. The compositions may further comprise one or more excipients, such as a co-solvent. The nicotine, organic acid, and/or any excipient may be dissolved in or may be suspended in the propellant. The formulation may be contained within a metered dose inhaler canister equipped with a metered chamber.

The present invention also provides an article of manufacture comprising a metered dose inhaler system. The metered dose inhaler system typically comprises, a canister, comprising an aerosol solution formulation of the present invention under appropriate pressure, a metering valve, and an actuator. One embodiment of this aspect of the invention comprises a sealed canister comprising the pharmaceutical formulation described above. In one version, the formulation comprises a substantially homogeneous solution formulation in the metered dose inhaler canister. The aerosol solution formulation in the metered dose inhaler canister typically comprises substantially a single-phase solution. Alternatively, the formulation may comprise one or more components that are suspended in a propellant or other liquid carrier.

The present invention also provides a method of making the pharmaceutical formulation and/or article of manufacture of the present invention. In one embodiment, a method of making the pharmaceutical compositions of the present invention comprises combining, to form an aerosol solution formulation, (i) free-base alkaloid, e.g., nicotine, (ii) an organic acid, wherein (a) the organic acid and the free-base alkaloid form a salt, and (b) an equivalent mixture of free-base alkaloid and organic acid in water has a pH between about pH 3.0 and about pH 9.0, and (iii) a hydrofluoroalkane propellant. A method of manufacturing a metered dose inhaler of the present invention may comprise, filling a canister under pressure with an appropriate amount of an aerosol solution formulation of the present invention, and sealing the canister.

The present invention also provides a method of administering the pharmaceutical formulation of the present invention to a user, such as a human. According to a version of the invention, a formulation comprising nicotine and organic acid is aerosolized and delivered to the respiratory tract of a user. In one version, the a metered dose inhaler is used to aerosolize the formulation. The administration may provide for treatment of a condition in the subject, for example, addiction to the alkaloid, suppressing appetite, neurological disorders, pain management, and/or use as an anti-inflammatory.

These features of the invention are described herein below with reference to nicotine as an exemplary alkaloid. These examples are not intended to be limiting. Other features may be apparent to one of ordinary skill upon reviewing the following specification and any attached claims.

In one exemplary embodiment, the present invention comprises a pharmaceutical formulation solution comprising nicotine or a derivative thereof. In one version, free-base nicotine is combined with an organic acid. Typically, the organic acid is present in a mole ratio with the nicotine in a range of about 0.25:1 (organic acid:nicotine) to about 4:1 (organic acid:nicotine), preferably in a range of about 0.5:1 (organic acid:nicotine) to about 2:1 (organic acid:nicotine), more preferably in a range of about 1:1 (organic acid:nicotine) to about 1.5:1 (organic acid:nicotine). The organic acid and free-base nicotine combine in solution to form a nicotine salt. Typically, an equivalent mixture of organic acid and free-base nicotine in water has a pH between about pH 3.0 and about pH 9.0, preferably between about pH 3.5 to about pH 7.5, more preferably between about pH 4.5 to about pH 7.4, most preferably about pH 6.8 to about pH 7.4. The organic acid and free-base nicotine may be combined in a co-solvent, for example, ethanol, before the addition of a hydrofluoroalkane propellant. Alternatively, they may be combined directly in the propellant in the presence or absence of a co-solvent. The use of ethanol as a co-solvent is described for the formulations set forth in Examples 1 and 5. The order of addition of the components of the aerosol solution formulations of the present invention may be empirically determined following the guidance of the present specification, in order to obtain formulations with the desired aerodynamic properties.

In one version, aerosol solution formulations of the present invention may comprise nicotine, organic acid and propellent, wherein about 0.01 to about 5 weight percent of the three components is nicotine, about 0.01 to about 5 weight percent of the three components is organic acid; and about 99.98 to about 90 weight percent of the three components is propellant. The formulation may further comprise additional components. When the aerosol solution formulations further comprise a co-solvent, aerosol solution formulations of the present invention may comprise, about 0.01 to about 5 weight percent of the four components of nicotine, about 0.01 to about 5 weight percent of the four components of organic acid, about 99.97 to about 75 weight percent of the four components of propellant; and about 0.01 to about 15 weight percent of the four components of co-solvent.

Numerous suitable organic acids may be used in the formulations of the present invention, including, but not limited to the following carboxylic or dicarboxylic acids: formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, tartaric acid, bitartaric acid, lactic acid, malonic acid, succinic acid, fumaric acid, finnaric acid, gluconic acid, saccharic acid, malonic acid, and malic acid. One or more organic acids may be combined in the formulations of the present invention. A preferred range of pKa's for organic acids for use in the present invention is a pKa of about 3 to a pKa of about 6. For example, the pKa propionic acid is about 5 and the pKa of lactic acid is about 3.8. Propionic acid and lactic acid were used to generate the formulations described in Examples 1 and 5.

Other compounds containing one or more functional COOH groups may be used as organic acids in the formulations of the present invention, for example, wherein the organic acid comprises polyethylene glycol (PEG) (e.g., mono-functionalized PEGs, such as polyethylene glycol-propionic acid). Such compounds may include polymers, copolymers, or terpolymers having at least one functional carboxyl group. A wide range of molecular weights of such compounds may be employed, for example, polyethylene glycol having an average molecular weight of between about 200 and about 1000. An aerosol solution formulation using polyethylene glycol having an average molecular weight of about 550 was generated following the methods of the present invention.

The formulations of the present invention may comprise more than one form of nicotine and/or its derivatives. For example, nicotine content may be formulated to correspond to that found in tobacco plants, e.g., nicotine accompanied by small amounts of nicotimine, nicoteine, and nicotelline.

A number of suitable co-solvents can be used in the formulations of the present invention including, but not limited to, the following: ethyl alcohol, isopropyl alcohol, n-propane, n-butane, isobutane, n-pentane, iso-pentane, neo-pentane, n-hexane, diethyl ether, propylene glycol, polyethylene glycol, polypropylene glycol, glycol ethers, glycerol, polyoxyethylene alcohols, and polyoxtethylene fatty acid esters. Mixtures of two or more co-solvents may be used as well. Typically the co-solvent is an alcohol acceptable for pharmaceutical use, for example, propanol, isopropanol, and/or ethanol.

A number of suitable propellants may be used in the formulations of the present invention. Preferred propellants include those of the hydrofluorocarbon (e.g., hydrofluoroalkanes) family, which are considered more environmentally friendly than the chlorofluorocarbons. Exemplary hydrofluoroalkanes include, but are not limited to, 1,1,1,2-tetrafluoroethane (HFC-134(a)), 1,1,1,2,3,3,3,-heptafluoropropane (HFC-227), difluoromethane (HFC-32), 1,1,1-trifluoroethane (HFC-143(a)), 1,1,2,2-tetrafluoroethane (HFC-134), 1,1-difluoroethane (HFC-152a), as well as combinations thereof. Particularly preferred are 1,1,1,2-tetrafluoroethane (HFC-134(a)), 1,1,1,2,3,3,3,-heptafluoropropane (HFC-227), and combinations thereof.

In one version of the present invention, greater than 50% of the free base nicotine is converted to a nicotine salt in combination with the organic acid. In another version, greater than about 80%, greater than about 90%, greater than about 95%, or greater than about 98% of the free base nicotine is converted to a nicotine salt in combination with the organic acid.

Canisters comprising the solutions of the present invention may have multiple phases, for example, a vapor phase, a solution phase, and a low-level particulate phase. In one version, there is substantially no vapor phase as the contents are maintained under pressure. Also, in one version of the formulation, the solution comprises less than about 10% non-dissolved particles. In another version, the solution is substantially free of non-dissolved particles. Solutions with low levels or the absence of non-dissolved particles are preferred because such formulations tend to provide more accurate and reproducible delivery nicotine and require little or no shaking that may be required for suspension of particles in the solution. Similarly, it is preferable that an aerosol solution formulation of the present invention is a substantially homogeneous solution and that the solution comprises a substantially single-phase solution. The formulations described in Examples 1 and 5 are substantially single-phase solutions. Further, the formulations described in Examples 1 and 5 present examples of substantially homogenous solutions.

In one version, the formulation is substantially free of added water. In another version, the formulation may be substantially free of a surface-active substance (i.e., a surfactant), such as a detergent or soap, that lowers the surface tension of a solvent. Further, mixtures of free-base forms of alkaloids in addition to or other than nicotine may be employed in the compositions, methods, and articles of manufacture of the present invention. In a particular embodiment of the present invention, the aerosol solution formulations consist essentially of free-base nicotine, an organic acid, wherein (a) the organic acid is present in a mole ratio with the nicotine in a range of about 0.25:1 (organic acid:nicotine) to about 4:1 (organic acid:nicotine), (b) the organic acid and the free-base nicotine form a nicotine salt, and (c) an equivalent mixture of free-base nicotine and organic acid in water has a pH between about pH 3.0 and about pH 9.0, and a hydrofluoroalkane propellant. In a related embodiment, the aerosol solution formulations consist essentially of free-base nicotine, an organic acid, wherein (a) the organic acid is present in a mole ratio with the nicotine in a range of about 0.25:1 (organic acid:nicotine) to about 4:1 (organic acid:nicotine), (b) the organic acid and the free-base nicotine form a nicotine salt, and (c) an equivalent mixture of free-base nicotine and organic acid in water has a pH between about pH 3.0 and about pH 9.0, a co-solvent, and a hydrofluoroalkane propellant.

Example 1 describes two exemplary aerosol solution formulations of the present invention, Nicotine Lactate and Nicotine Propionate solutions. The formulation of a third solution, Nicotine Free-Base, is described as well. The components in Table 1 were mixed in the following order. In Example 1, the small organic acid (e.g., 1-lactic acid or propionic acid) was dissolved in a quantity of ethanol at room temperature. The nicotine free-base was then dissolved in this solution. Solution formulations for use in MDIs were prepared by weighing the formulation components in a tared aluminum canister which was then sealed with a metering valve. Suitable canisters and metering valves are commercially available. Using an automated pressurized-liquid HFA metering system (commercially available) the sealed canister was filled through the valve stem with the required volume of liquid propellant. Three further nicotine lactate formulations were generated (Example 5) by increasing the total amount of ethanol co-solvent in the nicotine lactate formulation described in Example 1 from a total of 1% w/w ethanol up to 2%, 4%, and 9% w/w ethanol.

The aerosol solution formulations of the present invention are employed in inhalation methods. The aerosol solution formulations are typically packaged in metered dose inhalers, as described above. Metered dose inhaler devices typically comprise a canister, a metered dose valve and valve actuator, and a mouthpiece. The individual components are commercially available from a number of sources, for example, from Valois Pharmaceutical Division (Marly-le-Roi, France) or 3M Worldwide (3M General Offices, St. Paul, Minn.). For MDIs of the present invention, the canister is filled with a formulation according to the invention. The canister, which may be metal, keeps the medication under pressure. When the actuator is depressed a metered dose of the nicotine/organic acid is aerosolized for inhalation. Particles comprising the nicotine/organic acid are aerosolized in a form where they may be inhaled by a user.

The MDIs of the present invention may deliver, for example, a single metered dose of nicotine per administered aerosol puff of between about 20 μg and about 400 μg of nicotine, preferably between about 40 μg and about 100 μg are delivered per single metered dose, and more preferably between about 50 μg and about 80 μg are delivered per single metered dose. Typically about 50-80 μg of nicotine are delivered per puff by an average cigarette, with about 10 puffs per cigarette.

The MDIs comprising the aerosol solution formulations of the present invention are designed to typically deliver particles having a mass median aerodynamic diameter (MMAD) of particles comprising nicotine of less than 6.0 μm, preferably from 0.5 μm to 5.0 μm, more preferably from 1.0 μm to 4.0 μm, more preferably from 1.0 μm and about 3.0 μm. In preferred aerosol solution formulations, a fine particle dose percent of less than 4.7 μm particles (FPD-<4.7 μm) comprising nicotine, delivered by a metered dose inhaler, is between about 30% to about 90%. More preferably greater than or equal to about 50% of the nicotine in a single metered dose is delivered to the lungs in a fine particle dose percent of less than 4.7 μm particles comprising nicotine. Further, in preferred aerosol solution formulations, less than or equal to 30% (e.g., between about 5% to about 30%) of the nicotine in a single metered dose is deposited in the oropharyngeal region (i.e., throat). More preferably, less than about 15% of the nicotine in a single metered dose is deposited in the oropharyngeal region (i.e., throat). Example 1 describes methods of evaluating MMAD and FPD<4.71 μm.

In one aspect of the present invention, the delivery of nicotine using the MDIs of the present invention is desired to mimic the delivery of nicotine from smoking a cigarette. Accordingly, MDIs comprising the aerosol solution formulations of the present invention have been designed to provide pulmonary delivery of nicotine and may, for example, be used in treatment methods of smoking cessation in humans. The pharmacokinetics properties of nicotine delivery using the MDIs of the present invention follow the pharmacokinetic properties of nicotine delivered by smoking a cigarette. The formulations described herein allow the minimization of throat deposition of nicotine which will potentially increase acceptance by a patient being treated using the methods of the present invention. The MDIs comprising the aerosol formulations of the present invention have the following desirable attributes for a nicotine inhalation product: taste tolerability, appropriate pK profiles, stability, safety, simplicity, and the products are relatively inexpensive to produce.

Andrus, P. G., et al., (Can Respir J 6(6):509-512, 1999) describe a nicotine microaerosol inhaler. The reference describes the measurement of the droplet size distribution of a nicotine pressurized metered dose inhaler using nicotine in ethanol solution formulation with hydrofluoroalkane as propellant. This reference, however, describes only the use of free-base nicotine resulting in a formulation having a very basic pH. The reference neither teaches nor suggests the addition of organic acids as described in the aerosol solution formulations of the present invention. Example 1 describes a free-base nicotine formulation (“Nicotine Free Base”) similar to that described in the reference Andrus, et al. Example 1 also describes aerosol solution formulations of the present invention comprising organic acids (“Nicotine Lactate” and “Nicotine Propionate”). Example 2 presents data regarding the evaluation of several attributes that are relevant to pulmonary delivery of nicotine useful, in particular, in smoking cessation programs. The formulations were tested for aerosolization efficiency, FPD<4.7 μm, and throat deposition. Example 2 compares the Nicotine Free Base formulation with the Nicotine Lactate formulation of the present invention. The nicotine/organic acid formulation of the present invention (e.g., Nicotine Lactate formulation) was shown to have superior performance for the evaluated attributes than the Nicotine Free Base formulation.

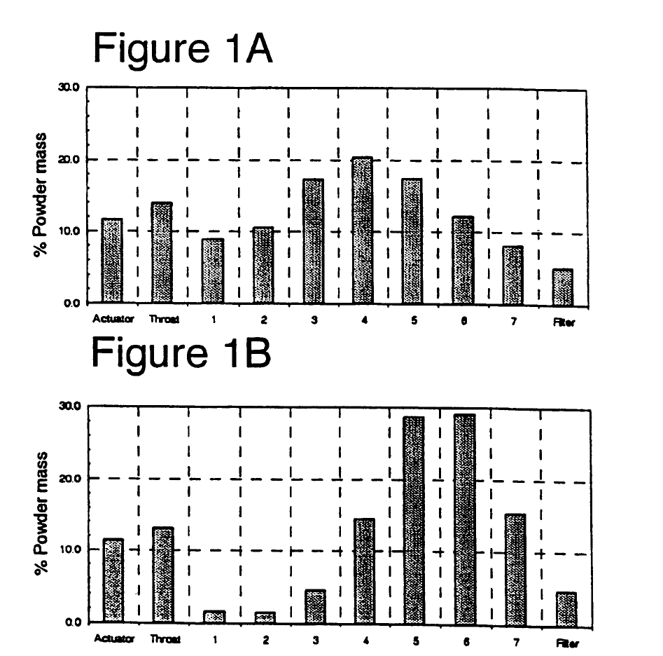

In Example 3 the Nicotine Lactate and Nicotine Free Base formulations described in Example 1 were evaluated further for properties related to aerosolized, pulmonary delivery of nicotine (e.g., MMAD, throat deposition, and respirable dose). The data demonstrated that the Nicotine Lactate formulation (an example of the nicotine/organic acid formulations of the present invention) had a more desirable MMAD size, lower throat deposition, and a higher respirable dose than the Nicotine Free Base formulation. Further, the skew of the deposition curve centered between stages 5 and 6 for the nicotine lactate formulation (FIG. 1B), versus being centered around stage 4 for the nicotine free base formulation (FIG. 1A), indicated that the nicotine lactate formulation provides a better (i.e., larger) FPD than the nicotine free base formulation. This result suggests better pulmonary delivery of nicotine by the nicotine/organic acid formulations of the present invention than by the nicotine free base formulation using MDIs.

Example 5 presents data concerning dose per puff of inhalation, actual percent recovery of nicotine, MMAD, FPD, and throat deposition for a number of formulations including Nicotine Free Base, Nicotine Propionate, Nicotine Lactate 1% w/w ethanol, Nicotine Lactate 2% w/w ethanol, Nicotine Lactate 4% w/w ethanol, and Nicotine Lactate 9% w/w ethanol. The results suggested that all of the nicotine lactate formulations were preferable to the nicotine propionate formulation, which was more preferable than the nicotine free base formulation. These results support the desirability of use of the nicotine/organic acid formulations of the present invention for use in therapeutic administration of nicotine to subjects. Further, the results demonstrated the superior properties of the nicotine/organic acid formulations of the present invention versus formulations with nicotine free base only. The data presented in FIGS. 3A, 3B, and 3C suggested that relatively lower amounts of ethanol (e.g., ethanol levels less than about 4% w/w) as co-solvent provide MDI nicotine/organic acid formulations (e.g., nicotine lactate formulations) that have more desirable therapeutic delivery properties.

The present invention also includes methods of making (i.e., methods of manufacturing) the aerosol compositions and metered dose inhalers described herein. For example, a method of making an aerosol solution formulation of the present invention may comprise combining (i) free-base nicotine, (ii) an organic acid, and (iii) a hydrofluoroalkane propellant. Such a method may further comprise combining the free base nicotine and organic acid in a co-solvent prior to addition of the propellant. The order of addition of the components may be empirically determined by one of ordinary skill in the art in view of the teachings of the present specification. A canister may be filled under pressure with the aerosol solution formulation of the present invention and the canister sealed. The canister may be sealed, for example, by crimping or by use of a metering valve. Further components of a metered dose inhaler system may be provided, for example, an actuator.

A number of commercial devices may be used for filling the canisters, for example, an automated pressurized-liquid HFA metering system. Sealed canister may, for example, be filled through the valve stem with the required volume of propellant.

Typically the canisters yield about 200 to 400 puffs per canister at a nominal unit dose of between about 20 μg/puff and about 400 μg/puff; these are nominal doses and the doses may be as high as about 800 puffs per canister. A single cigarette puff typically has approximately 100 μg/puff, based on 4 mg of nicotine per cigarette with an average of 20 puffs per cigarette, and 50% of the dose delivered to the lung. The weight percent of nicotine can be varied, for example, to provide a range of MDIs that deliver difference nicotine concentrations (e.g., 0.01% w/w, 0.1% w/w, and 1% w/w) to aid in smoking cessation programs. Typically, preferences and satisfaction with regard to harshness, strength, and similarity to cigarettes tend to increase proportionally with the percentage increase in nicotine.

The aerosol solution formulations of the present invention are employed in inhalation methods. Another aspect of the present invention comprises a method of treating a condition responsive to treatment by a nicotine/organic acid aerosol solution formulation, which method comprises pulmonarily administering to a subject in need thereof a physiologically effective amount of nicotine that comprises a therapeutically effective amount of nicotine. A variety of conditions may be treated by the compositions of the present invention including, but not limited to, treating nicotine addiction, suppressing appetite, preventing weight gain, treating neurological disorders (e.g., Parkinson's disease, Alzheimer's dementia, Tourette's syndrome, sleep apnea, attention deficit disorder, and pain relief), and use as an anti-inflammatory.

It has also been discovered that the formulation of the present invention provides a more palatable delivery of nicotine. Free-base nicotine has a harsh, unpleasant taste. In contrast, nicotine salt forms are less harsh and have a less unpleasant taste. In addition, free-base nicotine can sometimes lead to gastrointestinal upset more often than the salt form.

The physiologically effective amount needed to treat a particular condition or disease state will depend on the individual, the condition, length of treatment, the regularity of treatment, the type of drug, and other factors, but can be determined by one of ordinary skill in the medicinal arts in view of the teachings of the present specification.

In a general embodiment the present invention describes of method of administering nicotine to a subject (e.g., a human). The method typically comprises inhaling an aerosol solution formulation of the present invention from a metered dose inhaler, wherein the inhalation provides a pharmaceutically acceptable dose of nicotine to said subject. The formulations of the present invention can provide pulmonary delivery of nicotine to the subject.

In one embodiment the present invention includes a method of treating nicotine addiction in a subject. The method typically comprises inhaling an aerosol solution formulation of the present invention from a metered dose inhaler, wherein the inhalation provides a pharmaceutically acceptable dose of nicotine to said subject. In this embodiment of the invention a series of MDIs may be used, for example, wherein each MDI has a decreasing amount of nicotine delivered per puff in order to wean a subject away from dependence on nicotine. The weight percent of nicotine can be varied, for example, to provide a range of MDIs that deliver difference nicotine concentrations (e.g., 0.01% w/w, 0.1% w/w, and 1% w/w) to aid in smoking cessation programs. Typically, preferences and satisfaction with regard to harshness, strength, and similarity to cigarettes tend to increase proportionally with the percentage increase in nicotine.

Example 4 presents data from nicotine titration curves that were generated for the nicotine lactate and the nicotine propionate formulations presented in Example 1 (i.e., exemplary nicotine/organic acid solution formulations of the present invention). In titration experiments comparable amounts of nicotine and organic acid are combined in water as are used in the hydrofluoroalkane aerosol solution formulations described herein. In this way an equivalent pH value can be determined for the aerosol formulation. Typically the titration was carried out as follows. Nicotine was dissolved in water and the acid was prepared in water. Aliquots of acid were added to the nicotine solution and pH was measured at each point.

The data presented in Example 4 indicated that at an approximately 1.2:1 ratio (acid:nicotine) the majority of the nicotine free base was converted to the nicotine salt. These results demonstrate a desirable feature of the present invention, in that, when the organic acid/nicotine salts of the present invention are delivered by inhaled dose there is no dumping of strongly basic nicotine into the lungs, nor is there dumping of free acid into the lungs; rather, nicotine is delivered as a salt comprising nicotine and the associated organic acid. This is a highly desirable feature of the present invention as the present invention provides nicotine in an inhalable form that is more biocompatible than delivery of the free base and provides delivery of the nicotine in a pH range more acceptable to a subject, for example, a human.

Another feature for an acceptable delivery device of nicotine to humans, for use in smoking cessation programs, is a similarly between the pharmacokinetic profiles of delivery by the device and delivery by cigarette smoking. A human physiologically based pharmacokinetic (PBPK) model for nicotine disposition has been developed and tested in Sprauge Dawley (SD) rats (see, for example, Robinson D. E. et al., J Pharmkin Biopharm 20(6):591 (1992), and Plowchalk D. R. et al., Toxicol. Appl. Pharmacol 116(2):177 (1992)). Example 6 presents data regarding the pharmacokinetic profile of a nicotine formulation of the present invention relative to delivery of nicotine via smoking a cigarette, nasal delivery of nicotine, chewing gum (i.e., oral) delivery of nicotine, transdermal patch delivery of nicotine, and delivery of nicotine via MDIs comprising the aerosol solution formulations of the present invention.

The results of this demonstrated a high efficacy of nicotine delivery and venous plasma pharmacokinetics similar to that obtain when nicotine was introduced via smoking of a cigarette when nicotine was introduced via MDIs comprising the aerosol solution formulations of the present invention.

The compositions, devices, and methods of the present invention meet one or more of the following criteria for delivery of nicotine to a subject: (i) delivery of a precise dose of nicotine to the lungs, that is, accurate and reproducible delivery of specified doses, (ii) a palatable delivery of nicotine—free-base nicotine has a harsh, unpleasant taste and the tendency to lead to gastrointestinal upset, (iii) nicotine penetration into the lungs that simulates the sensation normally provided by nicotine when delivered by smoking a cigarette yet without the disadvantages of inhalation of combustion products from tobacco, (iv) methods and devices that are safe, both to the user and the environment, (v) methods and devices that are easy to use, and (vi) a pharmacokinetic profile resembling that of cigarette smoking, that is a profile that mimics the blood levels achieved by cigarette smoking by providing a sharp initial rise in blood level followed by a slow release of nicotine. The devices and methods of the present invention, as described herein, meet all of these criteria.

The following examples are put forth so as to provide those of ordinary skill in the art with a complete disclosure and description of how to make and use a particular and non-limiting form of the devices, methods, and formulae of the present invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet or are expected to meet the strict specifications for content and purity required of pharmaceutical products.

### EXAMPLE 1

### Exemplary Nicotine Formulations

This example describes several exemplary nicotine-containing, solution-based formulations for use in metered dose inhalers (MDIs).

Table 1 presents the proportion of components in the solution-based formulations. Pharmaceutical-grade components were used. The sources of the components were as follows: nicotine free-base (Siegfried, Switzerland), 1-lactic acid (Sigma-Aldrich, St. Louis Mo.) propionic acid (Sigma-Aldrich St. Louis Mo.), 1,1,1,2-tetrafluoroethane (HFA 134a; Dupont, Wilmington Del.), and dehydrated ethanol (Spectrum Chemical, Gardenia Calif.).

TABLE 1

| | Amount of | | Weight Percent | Amount of | | Weight Percent | Amount of | Weight Percent |
|---|---|---|---|---|---|---|---|---|
| | components | | (w/w) of | components | | (w/w) of | components | (w/w) of |
| | (in mg) in | | components | (in mg) | | components | (in mg) in | components |
| | final | | in final | in final | | in final | final | in final |
| | composition | | composition | composition | | composition | composition | composition |
| | “Nicotine | | “Nicotine | “Nicotine | | “Nicotine | “Nicotine | “Nicotine |
| Component | Lactate” | | Lactate” | Free Base” | | Free Base” | Propionate” | Propionate” |
| Nicotine | 16.9 | | 0.13% | 21.3 | | 0.2% | 16.7 | 0.2% |
| free-base | | | | | | | | |
| 1-lactate | 20.8 | | 0.16% | 0 | | 0 | 0 | 0 |
| propionate | 0 | | 0 | 0 | | 0 | 16.7 | 0.13% |
| HFA 134a | 12.5 | g | 98.4% | 10.3 | g | 97.6% | 12.9 | 99.7% |
| Ethanol | 84.2 | | 1.3% | 259.4 | | 2.4% | 0 | 0 |

The components in Table 1 were mixed in the following order. The small organic acid (e.g., 1-lactic acid) was dissolved the a quantity of ethanol at room temperature. The nicotine free-base was then dissolved in this solution (or alone in ethanol for the Nicotine Free Base formulation).

Solution formulations for use in MDIs were prepared by weighing the formulation components in a tared aluminum canister (Presspart Blackburn England). The canister was then sealed with a 25 or 50 μl metering valve (Valois, France; or 3M, Worldwide). Using an automated pressurized-liquid HFA metering system manufactured by Pamasol (Switzerland), the sealed canister was filled through the valve stem with the volume of liquid HFA-134a.

The canisters were then placed in a standard QVAR actuator (IVAX Laboratories, Miami Fla.) with an atomization orifice diameter of 0.27 mm. This yielded about 200 to 400 puffs per canister at a nominal unit dose of approximately 116 μg/puff for the Nicotine Free Base formulation, and 80 μg/puff for the Nicotine Lactate formulation; these are nominal doses, the doses may be as high as 800 puffs per canister. A single cigarette puff typically has approximately 100 μg/puff, based on 4 mg of nicotine per cigarette with an average of 20 puffs per cigarette, and 50% of the dose delivered to the lung.

The particle size distribution of the dose produced from the MDIs was assessed by firing a suitable number of shots into the Next Generation Impactor (NGI; MSP Corporation, Shoreview, Minn.), multi-stage cascade impactor, fitted with a USP 23 induction port and operated at a flow rate of 28.3 l/min. The components (including each of the stages) of the apparatus were coated with citrate buffer at pH 3.4 which was mixed at 50% v/v with glycerin to minimize loss of high-volatile nicotine. The drug deposited on each component of the apparatus was recovered by washing with 100 mM citrate buffer at pH 3.4 with no glycerin and quantified using a UV spectrophotometric method. The mass median aerodynamic diameter (MMAD) and geometric standard deviation (GSD) were calculated following the manufacturer's instructions.

The fine particle dose (FPD), equivalent to the mass of particles less than 4.7 μm per actuation, was calculated from the total drug deposited on stages 3 to the filter of the multi-stage cascade impactor.

For each sizing experiment 10 doses were discharged at 10 second intervals into a USP throat attached to the Next Generation Impactor. Sizing was performed at room temperature.

### EXAMPLE 2

### Comparisons of Solution Based and Suspension Based Formulations

Two of the formulations presented in Example 1 were evaluated with regard to several attributes that are relevant to pulmonary delivery of nicotine useful, in particular, in smoking cessation programs. The formulations were tested for aerosolization efficiency, FPD-<4.7 μm, and throat deposition.

Table 2 presents a relative comparison of the results of comparisons of these attributes for the two formulations.

TABLE 2

| | Nicotine | |
|---|---|---|
| | Lactate | Nicotine Free |
| Attribute | Solution | Base Solution |
| Efficiency ≧ 50% | ++ | + |
| FPD < 4.7 μm = | ++ | + |
| 50-80 μg | | |
| <30% Throat Deposition | +++ | ++ |

In Table 2, the plus symbols (+) designate relative levels of success between the two formulations tested.

As can be seen by the above comparisons, the nicotine lactate solution had the most desirable combination of attributes. Accordingly, the nicotine lactate solution (an example of nicotine in combination with an organic acid) appeared to overall have the most desirable properties for use in smoking cessation programs.

### EXAMPLE 3

### Comparisons of MMAD, Throat Deposition, and Resipirable Dose

The nicotine lactate and nicotine free base formulations described in Example 1 were evaluated further for properties related to aerosolized, pulmonary delivery of nicotine. MMAD was evaluated as described in Example 1. Throat deposition corresponds to the amount of nicotine deposited in the USP throat attached to the NGI device and was evaluated as described in Example 1. Respirable dose corresponds to the total amount of nicotine deposited on stages 3-end filter of the NGI device and was evaluated as described in Example 1.

Table 3 presents a summary of the data that was obtained in this study.

TABLE 3

| | Nicotine Free Base | Nicotine Lactate |
|---|---|---|
| | Formulation | Formulation |
| | (HFA 134a; 116 | (HFA 134a; |
| Properties | μg/puff) | 80 μg/puff) |
| MMAD (μm) | 2.8 | 1.4 |
| Throat Deposition | 31.5/27% | 12.5/15% |
| (μg/percent of total | | |
| delivered nicotine | | |
| dose) | | |
| Respirable Dose | 60/52% | 58/72% |
| (μg/percent of total | | |
| delivered nicotine | | |
| dose) | | |

As can be seen from the data in Table 3 the nicotine lactate formulation had a more desirable MMAD size (1.4 μm) than the nicotine free base formulation. Further, the nicotine lactate formulation had lower throat deposition (˜15% of total nicotine delivered dose) and provided a higher respirable dose (˜72% of the total nicotine delivered dose) than the nicotine free base formulation.

In addition, FIGS. 1A and 1B show the percent powder mass (vertical axis) deposited on the various stages of the NGI device using, respectively, the nicotine free base formulation and the nicotine lactate formulation. The skewed of the deposition curve centered between stages 5 and 6 for the nicotine lactate formulation, versus being centered around stage 4 for the nicotine free base formulation, indicated that the nicotine lactate formulation provided a better (i.e., larger) FPD than the nicotine free base formulation. This result suggested better pulmonary delivery of nicotine by the nicotine lactate formulation versus the nicotine free base formulation.

### EXAMPLE 4

### Nicotine Titration Curve

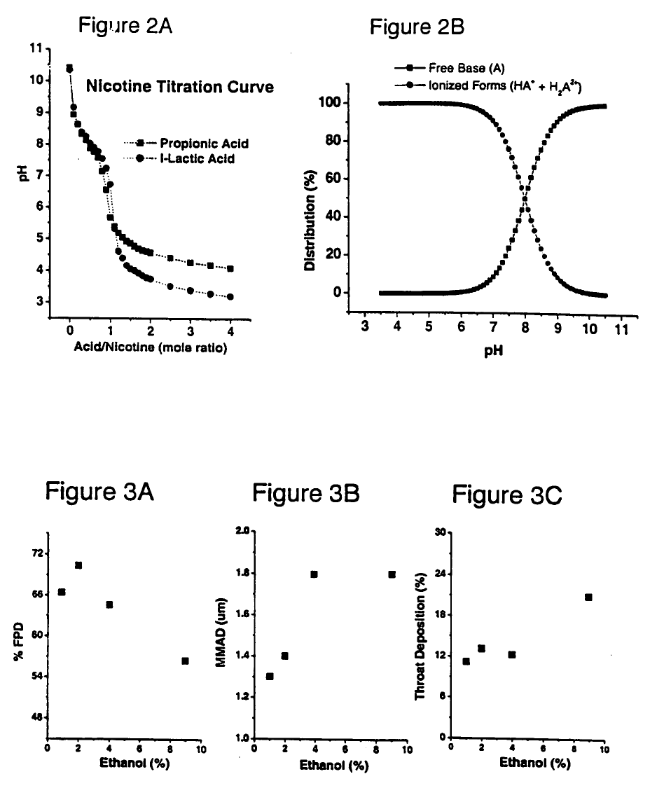

Nicotine titration curves were generated for the nicotine lactate and the nicotine propionate formulations presented in Example 1. Briefly, the titration was carried out as follows. 0.1M nicotine was dissolved in water and 1.0M lactic acid and propionic acid were prepared in water. 10 μl aliquots of acid were added to the nicotine solution and pH was measured at each point. The nicotine titration curves are presented in FIG. 2A. In the figure, the vertical axis is pH and the horizontal axis is the mole ratio of Acid/Nicotine.

The data from the nicotine titrations were then plotted as percent distribution between the free base and ionized forms of nicotine. This data is presented in FIG. 2B. In the figure, the vertical axis is the percent distribution of the form of nicotine (Free Base, A; Ionized forms HA⁺+H₂A²) and the horizontal axis is pH.

The results of this experiment indicated that at an approximately 1.2:1 ratio (acid:nicotine) the majority of the nicotine free base was converted to the nicotine salt. These results demonstrated a desirable feature of the present invention, in that, when the organic acid/nicotine salts of the present invention are delivered by inhaled dose there is no dumping of strongly basic nicotine into the lungs, nor is there dumping of free acid into the lungs; rather, nicotine is delivered as a salt comprising nicotine and the associated organic acid. This is a highly desirable feature of the present invention as the present invention provides nicotine in an inhalable form that is more biocompatible than delivery of the free base and provides delivery of the nicotine in a pH range more acceptable to a subject, for example, a human.

### EXAMPLE 5

### Formulation Related Aerosol Properties

In addition to the nicotine free base, nicotine lactate, and nicotine propionate formulations described in Example 1, three further nicotine lactate formulations were generated by increasing the total amount of ethanol co-solvent in the nicotine lactate formulation described in Example 1 (which had a total of 1% w/w ethanol) as follows: 2%, 4%, and 9% w/w.

The dose per puff of inhalation, actual percent recovery of nicotine, MMAD, FPD, and throat deposition were determined essentially as described above in Examples 1 or by standard methods. The results from these determinations are presented in Table 4. All values in the table are the average of two sets of measurements (i.e., each sample was tested twice).

TABLE 4

| | | Percent | | | Throat |
|---|---|---|---|---|---|
| Formulation | Dose/ | Recovery | MMAD | FPD % < | Deposition |
| Composition | puff | (%) | (μm) | 4.7 μm | (%) |
| Nicotine free | 115.6 | 105.7 | 2.9 | 52.4 | 12.7 |
| base | | | | | |
| (HFA134a) | | | | | |
| Nicotine | 124.1 | 75.9 | 2.5 | 54.6 | 8.7 |
| propionate | | | | | |
| (HFA134a) | | | | | |
| Nicotine | 70.7 | 93.9 | 1.3 | 66.2 | 11.2 |
| lactate, 1% | | | | | |
| ethanol | | | | | |
| (HFA134a) | | | | | |
| Nicotine | 79.9 | 101.5 | 1.4 | 71.3 | 13.1 |
| lactate, 2% | | | | | |
| ethanol | | | | | |
| (HFA134a) | | | | | |
| Nicotine | 189.8 | 95.4 | 1.8 | 65.6 | 12.4 |
| lactate, 4% | | | | | |
| ethanol | | | | | |
| (HFA134a) | | | | | |
| Nicotine | 205 | 89.3 | 1.8 | 56.6 | 21.0 |
| lactate, 9% | | | | | |
| ethanol | | | | | |
| (HFA134a) | | | | | |

The results presented in Table 4 suggested that, with regard to desirable MMAD and FPD properties, all of the nicotine lactate formulations and nicotine propionate formulation were more preferable than the nicotine free base formulation. All of the nicotine lactate formulations (with ethanol concentrations ranging from 1% to 9% w/w) had MMAD values of 1.8 μm or less and FPD %<4.7 μm of 56 or greater. These results supported the desirability of the formulations of the present invention (versus formulations of free base nicotine in the absence of organic acid) for use in therapeutic administration of nicotine to subjects.

Further, the effect of ethanol (the co-solvent) on the aerosol properties of nicotine lactate MDI formulations was evaluated. When the data presented in Table 4 for the various nicotine lactate formulations is plotted for FPD %<4.7 μm versus percent of ethanol (FIG. 3A) it was seen that at higher percentages of ethanol in the composition the FPD %<4.7 μm decreased. When the data presented in Table 4 for the various nicotine lactate formulations was plotted for MMAD versus percent of ethanol (FIG. 3B) it was seen that as the percentage of ethanol increased in the composition the MMAD increased. When the data presented in Table 4 for the various nicotine lactate formulations was plotted for throat deposition (%) versus percent of ethanol (FIG. 3C) it can be seen that as the percentage of ethanol increased in the composition (in this case as it approaches 9%) the percent throat deposition increased. These results suggested that relatively lower amounts of ethanol as co-solvent provide MDI nicotine lactate formulations having more desirable therapeutic delivery properties (e.g., ethanol levels less than about 4% w/w).

### EXAMPLE 6

### Predicted Pharmacokinetic Profiles

A human physiologically based pharmacokinetic (PBPK) model for nicotine disposition has been developed and tested in Sprauge Dawley (SD) rats (see, for example, Robinson D. E. et al., J Pharmkin Biopharm 20(6):591 (1992), and Plowchalk D. R. et al., Toxicol. Appl. Pharmacol 116(2):177 (1992)).

The PBPK model successfully describes the tissue and plasma kinetics of nicotine in the SD rat and is a useful tool for pharmacologic studies in humans and experimental animals that require insight into the plasma or tissue concentration-effect relationship. In humans the main targeted compartments for nicotine are the lungs, arteries, brain, and veins when nicotine is administered by inhalation (for example, by cigarette smoking or MDI inhalation).

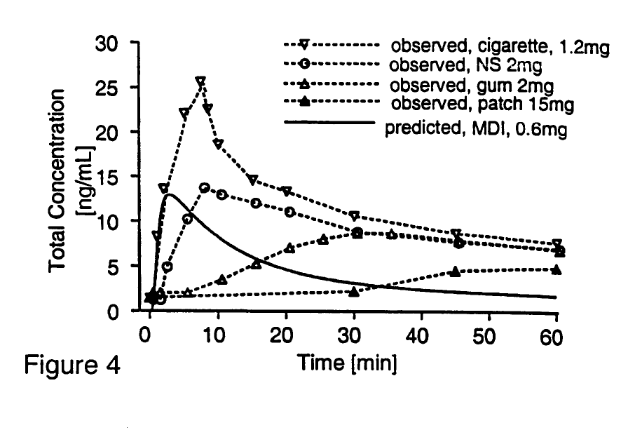

The pharmacokinetic profile of a MDI comprising a nicotine aerosol solution formulation of the present invention (FIG. 4, MDI 0.6 mg) having a 50% FPD %<4.7 μm, wherein the dose is 0.6 mg nicotine was evaluated using the PBPK model. The curve in FIG. 4 determined for “predicted MDI 0.6 mg” was generated as taught by the references of Robinson, et al., and Plowchalk, et al.

In FIG. 4, the dotted line with downward facing triangles shows the venous plasma concentration after smoking a cigarette delivering 1.2 mg of nicotine, the dotted line with open circles shows the venous plasma concentration after 2 mg of nicotine was delivered via Nasal route, the dotted line with upward facing triangles (hollow) shows the venous plasma concentration after delivery of 2 mg of nicotine in a gum-format (i.e., by chewing), the dotted line with upward facing triangles (solid) shows the venous plasma concentration over time with a nicotine patch comprising 15 mg of nicotine, and the solid line shows the predicted venous plasma concentration after 3 inhalations with a MDI of the present invention having 0.6 mg of nicotine that delivered 0.2 mg/puff.

This result with the MDI of the present invention comprising the nicotine and 1% ethanol formulation (Table 5, above) demonstrates high efficacy of nicotine delivery and venous plasma pharmacokinetics similar to that obtain when nicotine was introduced via smoking of a cigarette. These results support the usefulness and efficacy of delivering nicotine in a MDI using the formulations of the present invention.

Although the present invention has been described in considerable detail with regard to certain preferred versions thereof, other versions are possible. Various modification and variations of the above embodiments can be made without departing from the spirit and scope of this invention. For example, it is to be understood that this invention is not limited to particular types of metered dose inhalers, particular hydrofluoroalkane propellants, particular sources of alkaloids, e.g., nicotine, and the like, as use of such particulars may be selected in view of the teachings of the present specification. Furthermore, certain terminology has been used for the purposes of descriptive clarity, and not to limit the present invention. Therefore, the appended claims should not be limited to the description of the preferred versions contained herein and should include all such alterations, permutations, and equivalents as fall within the true spirit and scope of the present invention.

What is claimed is:

1. An aerosolizable formulation comprising:
free-base nicotine;
an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0; and
a hydrofluoroalkane propellant.

2. An aerosolizable formulation according to claim 1 wherein the formulation comprises about 0.01 to about 5 weight percent of nicotine; about 0.01 to about 5 weight percent of organic acid; and about 90 to about 99.98 weight percent of propellant.

3. An aerosolizable formulation according to claim 1 further comprising a co-solvent.

4. An aerosolizable formulation according to claim 3 wherein said co-solvent is selected from the group consisting of ethyl alcohol, isopropyl alcohol, n-propane, n-butane, isobutane, n-pentane, iso-pentane, neo-pentane, n-hexane, diethyl ether, propylene glycol, polyethylene glycol, polypropylene glycol, glycol ethers, glycerol, polyoxyethylene alcohols, and polyoxtethylene fatty acid esters.

5. An aerosolizable formulation according to claim 4 wherein said co-solvent is selected from the group consisting of propanol, isopropanol, and ethanol.

6. An aerosolizable formulation according to claim 4 wherein said co-solvent is ethanol.

7. An aerosolizable formulation according to claim 3 wherein the formulation comprises about 0.01 to about 5 weight percent of nicotine; about 0.01 to about 5 weight percent of organic acid; about 75 to about 99.97 weight percent of propellant; and about 0.01 to about 15 weight percent of co-solvent.

8. An aerosolizable formulation according to claim 1 wherein said organic acid is a carboxylic or dicarboxylic acid.

9. An aerosolizable formulation according to claim 8 wherein said organic acid is selected from the group consisting of formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, tartaric acid, bitartaric acid, lactic acid, malonic acid, succinic acid, fumaric acid, finnaric acid, gluconic acid, saccharic acid, malonic acid, and malic acid.

10. An aerosolizable formulation according to claim 9 wherein said organic acid is propionic acid or lactic acid.

11. An aerosolizable formulation according to claim 8 wherein said organic acid comprises polyethylene glycol.

12. An aerosolizable formulation according to claim 11 wherein said organic acid is polyethylene glycol-propionic acid.

13. An aerosolizable formulation according to claim 11 wherein said polyethylene glycol has an average molecular weight of between about 200 and about 1000.

14. An aerosolizable formulation according to claim 11 wherein said polyethylene glycol has an average molecular weight of about 550.

15. An aerosolizable formulation according to claim 1 wherein the pKa of said organic acid is about 3 and about 6.

16. An aerosolizable formulation according to claim 1 wherein said organic acid comprises more than one organic acid.

17. An aerosolizable formulation according to claim 1, wherein the mole ratio of organic acid to nicotine is between about 0.5 to about 2.0.

18. An aerosolizable formulation according to claim 1 wherein the mole ratio of organic acid to nicotine is between about 1.0 to 1.5.

19. An aerosolizable formulation according to claim 1 wherein the formulation comprises more than one form of nicotine.

20. An aerosolizable formulation according to claim 1 wherein said hydrofluoroalkane propellant is selected from the group consisting of 1,1,1,2-tetrafluoroethane (HFC-134(a)), 1,1,1,2,3,3,3,-heptafluoropropane (HFC-227), difluoromethane (HFC-32), 1,1,1-trifluoroethane (HFC-143(a)), 1,1,2,2-tetrafluoroethane (HFC-134), 1,1-difluoroethane (HFC-152a), and combinations thereof.

21. An aerosolizable formulation according to claim 20 wherein said hydrofluoroalkane propellant is selected from the group consisting of 1,1,1,2-tetrafluoroethane (HFC-134(a)), 1,1,1,2,3,3,3,-heptafluoropropane (HFC-227), and combinations thereof.

22. An aerosolizable formulation according to claim 1 wherein greater than about 50% of the free base nicotine is converted to a nicotine salt in combination with said organic acid.

23. An aerosolizable formulation according to claim 1 wherein greater than about 80% of the free base nicotine is converted to a nicotine salt in combination with said organic acid.

24. An aerosolizable formulation according to claim 1 wherein greater than about 90% of the free base nicotine is converted to a nicotine salt in combination with said organic acid.

25. An aerosolizable formulation according to claim 1 wherein greater than about 95% of the free base nicotine is converted to a nicotine salt in combination with said organic acid.

26. An aerosolizable formulation according to claim 1 wherein greater than about 98% of the free base nicotine is converted to a nicotine salt in combination with said organic acid.

27. An aerosolizable formulation according to claim 1 wherein the solution comprises less than about 10% non-dissolved particles.

28. An aerosolizable formulation according to claim 1 wherein the solution is substantially a single-phase solution.

29. An aerosolization apparatus comprising:
a canister comprising an aerosolizable formulation according to claim 1, said formulation being under pressure;
a metering valve, and
an actuator.

30. An aerosolization apparatus according to claim 29 wherein the aerosolizable formulation in said metered canister is a substantially homogenous solution.

31. An aerosolization apparatus according to claim 29 wherein the aerosolizable formulation in said metered canister is substantially a single-phase solution.

32. An aerosolization apparatus according to claim 29 wherein a single metered dose of nicotine per administered aerosol puff is between about 20 μg and about 400 μg of nicotine.

33. An aerosolization apparatus according to claim 29 wherein a mass median aerodynamic diameter of particles comprising nicotine is between about 1.0 μm and about 4.0 μm.

34. An aerosolization apparatus according to claim 29 wherein a fine particle dose percent of less than 4.7 μm of particles comprising nicotine is between about 30% to about 90%.

35. An aerosolization apparatus according to claim 29 wherein a percentage throat deposition of nicotine relative to the total dose of nicotine delivered per aerosol puff is less than about 30%.

36. A method of manufacturing an aerosolization apparatus for administering aerosolized nicotine to a user, the method comprising:
combining to form an aerosolizable formulation: (i) free-base nicotine, (ii) an organic acid, wherein (a) said organic acid is present in a mole ratio with said nicotine in a range of about 0.25:1 (organic acid:nicotine) to about 4:1 (organic acid:nicotine), (b) said organic acid and said free-base nicotine form a nicotine salt, and (c) an equivalent mixture of free-base nicotine and organic acid in water has a pH between about pH 3.0 and about pH 9.0, and (iii) a hydrofluoroalkane propellant;
filling a canister under pressure with an appropriate amount of said aerosolizable formulation; and
sealing said canister.

37. A method according to claim 36 further comprising inserting a metering valve in or near the canister.

38. A method according to claim 37 further comprising inserting an actuator on or near the metering valve.

39. A method according to claim 36 wherein said combining step further comprises adding a co-solvent to the aerosolizable formulation.

40. A method of treating nicotine addiction in a subject comprising:
aerosolizing an aerosolizable formulation according to claim 1; and
administering the aerosolized formulation to the respiratory tract of the subject during the subject's inhalation.

41. A method according to claim 40 wherein a dose of nicotine per administered aerosol puff is between about 20 μg and about 400 μg of nicotine.
